(19) United States
(12) Patent Application Publication
(10) Pub. No.: US 2013/0289368 A1
(43) Pub. Date: Oct. 31, 2013

(54) DIAGNOSTIC DEVICE

(71) Applicants: James Covington, Coventry (GB); Ramesh Arasaradnam, Coventry (GB); Chuka Nwokolo, Coventry (GB)

(72) Inventors: James Covington, Coventry (GB); Ramesh Arasaradnam, Coventry (GB); Chuka Nwokolo, Coventry (GB)

(73) Assignee: The University of Warwick, Warwickshire (GB)

(21) Appl. No.: 13/882,155

(22) PCT Filed: Oct. 24, 2011
(86) PCT No.: PCT/GB11/52063

§ 371 Date: Jul. 15, 2013

(30) Foreign Application Priority Data
Oct. 29, 2010 (GB) 1018253.3

(51) Int. Cl. A61B 5/07; A61B 1/04; A61B 5/145

(52) U.S. Cl. CPC A61B 5/073; A61B 5/14507; A61B 1/041
USPC 600/302; 600/309; 600/310; 600/109

(57) ABSTRACT

The invention relates to diagnostic devices, which are capable of characterising gases and other volatile organic compounds (VOCs) present in the gastrointestinal tract, for diagnosing diseases. The invention extends to apparatuses for use in the in vivo detection and characterisation of gases and VOCs, and to methods for diagnosing diseases.

The present invention relates to diagnostic devices, which are capable of characterising gases and other volatile organic compounds (VOCs) present in the gastrointestinal tract, for diagnosing diseases. The invention extends to apparatuses for use in the in vivo detection and characterisation of gases and VOCs, and to methods for diagnosing diseases.

The ability to diagnose and characterise a disease through the gaseous emissions of disease volatile organic compounds (dVOCs) from a patient is becoming increasingly recognised in medicine. Technical studies have shown that it is possible to detect, amongst others, melanoma, lung cancers, eye infections, brain cancers, schizophrenia, diabetes, wound infections, urinary tract infections and MRSA, from the gaseous emissions emanating from patient's blood, sweat, breath, urine and faecal samples. For example, in the detection of lung cancer, breath analysis is being used as an early biomarker. In addition, gaseous emissions can be detected from urine and faecal samples for the diagnosis of gastroenterological and metabolic diseases, such as diabetes, Crohn's and ulcerative colitis. Thus, the disease, or at least the host's reaction to the disease, can be identified through the gases dissolved within a patient's urine and faecal samples. In gastrointestinal diseases, dVOCs are disparate between inflammatory conditions compared with more benign ones, such as irritable bowel syndrome.

Importantly, dVOCs remain abnormal even when in clinical remission and do not return to normal as in the case of ulcerative colitis. Hence, a major problem with this approach is that, although it can be used to identify the type of disease and its severity, it provides almost no contemporaneous information as to the extent of activity and location of the disease. For example, it is not possible to determine if the disease is in a small part or all of the small bowel, or all or just a part of the large bowel, or both, using standard dVOC measurements. Furthermore, there is often a lag from clinical symptoms to presentation and production of a biological sample for analysis, and this can make clinical interpretation difficult, particularly with initiation of treatment.

Furthermore, it is known that it is also possible to identify bacterial diseases through the analysis of gaseous emissions from cultures grown from, for example, wound dressings. These gaseous emissions are either sampled directly from the patient, such as from sweat or breath, or the emission is captured, and then taken to a laboratory for processing and subsequent identification of the associated disease. This analytical process is normally performed using equipment such as Gas Chromatography/Mass Spectrometer (GC/MS), though other studies have used Selected Ion Flow Tube—MS (SIFT-MS), Fourier transform Infra-red spectrometry (FTIR) and Ion Mobility Spectrometry (IMS). Importantly, most emissions are specific to a disease group, and so it is possible to identify specific diseases. Unfortunately, these techniques require the analysis of biological samples from patients (e.g. faeces, urine or sweat etc.), which have inherent difficulties, including the difficulty for the patient to produce a usable sample on demand, as well as storage issues, and the time lost between sample collection and the analysis being carried out. Moreover, there is often a lag from clinical presentation to obtaining the actual biological sample, which may lead to lower concentrations of gases being emitted, which are therefore more difficult to detect.

There are several known methods for detecting dVOCs and gases from biological samples obtained from a patient. For example, gases and dVOCs can be detected using resistive metal oxide gas sensors/mixed metal oxide gas sensors, electrochemical gas sensors, optical/IR gas sensors, and conducting polymer/composite polymer resistive/capacitive gas sensors, quartz crystal microbalance gas sensors and pellister/calorimetric gas sensors.

A number of imaging techniques are available for diagnostic purposes. For example, these include endoscopic examinations, radiological tests, such as X-rays/CT (computer tomography) scans, MRI (magnetic resonance imaging) and ultrasound. Problems with these approaches however are that they are highly invasive and expose the patient to significant amounts of radiation, especially in the case of repeated imaging to assess disease progression over time (since some patients have intermittent imaging for over a half a century if diagnosed at a young age). Though successful, it can be difficult to specifically identify certain disease groups with these imaging methods, and the resolution of the images can be poor. Furthermore, these procedures use very expensive equipment, and so waiting times for imaging can be considerable. Moreover, confirming a diagnosis is often difficult and requires several modalities for confirmation, including clinical history, pathological review and radiological imaging.

Clearly, it is important for the patient, as well as the clinician, to be able to predict disease course and plan treatment accordingly. At present, it is possible to visually image most of the gastrointestinal tract using an endoscope, which involves a fibre optic system which illuminates and captures images from the body. However, there are three major problems using such an invasive technique. Firstly, its mucosal coverage for diagnosis is limited, especially in the small bowel which can extend up to 5 m in length. Modern endoscopes can visualise this segment, but the procedure requires considerable expertise, is time-consuming and is invasive for the patient. Secondly, although it is possible to visualise the diseased area, it still may not be possible to identify the disease without physically taking a biopsy, and even this, at times, can be inconclusive. Finally, there is also delay in obtaining a diagnosis using endoscopy, as it requires preparation in the lab and has added cost implications.

Pilot studies have been conducted which attempt to capture the gas emitted from a diseased area using an endoscope within a defined area of the gut, and then analysing those gases whilst the procedure is being undertaken. However, these studies were still unable to reach all regions of the gastrointestinal tract. Moreover, a significant limitation with this procedure is that the preparation required for performing diagnostic endoscopy alters the dVOC profile which it is designed to detect, thereby reducing the evidence which it was designed to identify in the first place. A further disadvantage of endoscopy is that it is uncomfortable to the patient, costly and delays treatment, due to the time required scheduling for the procedure.

In view of the foregoing, it is clear that there is a considerable need to provide improved apparatuses and methods for detecting and analysing dVOCs and gases emitted by patients, to facilitate quick and accurate diagnosis of disease. The inventors have therefore devised a novel device, which can be used to detect volatile organic compounds and gases for diagnosing diseases.

Thus, according to a first aspect of the invention, there is provided an ingestible diagnostic device comprising detection means for detecting gases and/or volatile organic compounds (VOCs).

Advantageously, the diagnostic device is ingestible, and so, in contrast to endoscopy, is non-invasive and does not alter the gas/VOC profile in the subject, which it is designed to detect for diagnosis of the disease. The device is preferably capable of detecting and characterising gases and other VOCs present in the gastrointestinal tract in vivo, for diagnosing disease. Thus, the gases and VOCs detected by the detection means have preferably been emitted by a subject who has ingested the device.

Examples of gases that may be detected by the detection means include nitrous oxide, hydrogen sulphide, carbon dioxide and hydrogen in concentrations of about 100 parts per million and below, in air. The skilled person will appreciate that volatile organic compounds (VOCs) can be organic chemical compounds, which have significant vapour pressure, and which can affect human or animal health. Examples of VOCs that may be detected include ethanoic, butanoic and pentanoic acids, benzaldehyde, ethanal, carbon disulfide, dimethyldisulfide, acetone, 2-butanone, 2,3-butanedione, 6-methyl-5-hepten-2-one, indole, and 4-methylphenol.

Advantageously, the device of the first aspect may be used to identify and provide diagnostic information relating to the type and/or severity of a wide variety of diseases by their gaseous/vapour emissions. By way of example only, the subject may suffer from gastroenteritis, which is inflammation of the gastrointestinal tract, often resulting in diarrhoea. The inflammation is frequently caused by an infection from certain viruses or bacteria, their toxins, parasites, or an adverse reaction to something in the diet or medication. At least 50% of cases of gastroenteritis due to foodborne illnesses are caused by norovirus, and another 20% of cases, and the majority of severe cases in children, are due to rotavirus infections. Other significant viral agents include adenovirus and astrovirus. Different species of bacteria can also cause gastroenteritis, including Salmonella, Shigella, Staphylococcus, Campylobacter jejuni, Clostridium, Escherichia coli, Yersinia, Vibrio cholerae, and others. Each organism causes slightly different symptoms, but all result in diarrhoea. Colitis, inflammation of the large intestine, may also be present.

Each of the above-mentioned micro-organisms is known to emit a signature of various gases and VOCs, and so the detection of certain gases and VOCs by the device of the first aspect is indicative of an infection with one or more of these micro-organisms. Once a clinician has determined that the subject has been infected by a certain micro-organism (e.g. virus, bacterium or fungus), it is then possible to diagnose a disease, and suggest a suitable treatment regime.

The detection means may comprise one or more chemical sensors, which are capable of detecting gas and/or VOCs emitted from the gastrointestinal tract of the subject. The gaseous/VOC emissions may be detected by the detection means using a variety of different technologies. For example, the detection means may detect gas and/or VOC using technology selected from the group consisting of: resistive metal oxide (e.g. doped/undoped SnO₂); resistive mixed metal oxide (e.g. combinations of SnO₂, WO₃ and/or ZnO, which may be mixed together to create a sensing layer); electrochemical sensors (e.g. through an oxidation/reduction reaction of the target gas on working electrodes); resistive/capacitive/frequency measurement of conducting polymers (e.g. polypyrrole or polyaniline doped with a counter ion of decane sulphonate (DSA) or butane sulphonate (BSA); resistive/capacitive/frequency measurement of composite polymers (e.g. carbon black nanoparticles dispersed in a polymer matrix of for example, polyethylene glycol); optical measurement using infra-red (e.g. LED or some other IR-source, light filter with a photodetector); frequency measurement quartz crystal micro-balances/shear horizontal surface acoustic wave sensors (e.g. lithium niobate or lithium tantalite, with and without a bio-sensing layer, for example a polymer or bio-coating); gas thermal measurement using pellisters/calorimetric (e.g. catalytic coating, such as palladium or platinum) of a bead formed from alumina oxide; and thermal measurement techniques using a thermal conductivity sensor and bio-sensor (e.g. an enzyme or protein attached to a secondary transducer).

The detection means may be internal or external. In one embodiment, the detection means may be disposed on or towards the surface of the device. The device may comprise a gas permeable membrane or layer, which substantially surrounds the detection means. The membrane is adapted to allow gas and VOCs to pass therethrough and reach the detection means, but prevents bodily fluids or solids from reaching the detection means. Advantageously, therefore, the gas permeable membrane provides an effective barrier to the bodily fluids or solids suspended therein, which could otherwise interfere with the accurate detection of the gases and VOCs in the tract, as the device passes therealong. The membrane may be porous. In use, gases are able to pass through the membrane into a small area inside the device where the sensors would interact with gas.

In another embodiment, the detection means may be disposed inside the device. The device may comprise at least one channel, a first end of which is connected to an aperture disposed on the outer surface of the device, and a second end of which is arranged such that it is at least adjacent the detection means. Thus, gas and VOCs emitted by the subject pass through the aperture and along the channel, such that it is fed to the detection means. The device may comprise a plurality of apertures and channels interconnecting the detection means.

The device may comprise position sensing means, which is capable of determining the location of the device when ingested by the subject, preferably as it passes along the gastrointestinal tract.

The device may comprise a camera, which is capable of taking still images and/or video footage, preferably in the gastrointestinal tract. The camera may use complementary metal oxide semi-conductor (CMOS) or charge-coupled device (CCD) camera technology, which may be illuminated by at least one white or blue LED. The camera may be capable of taking pictures and/or video either simultaneously or serially with the measurements of gas and/or VOC taken by the detection means.

The device may comprise means for detecting pH, preferably in the gastrointestinal tract. For example, the device may comprise a pH meter.

The device may comprise means for detecting temperature, preferably in the gastrointestinal tract. For example, the device may comprise a thermometer.

The device may comprise means for detecting dissolved oxygen concentration, preferably in the gastrointestinal tract. For example, the device may comprise a dissolved oxygen probe.

The device may comprise means for detecting thermal conductivity, preferably in the gastrointestinal tract. For example, the device may comprise a thermal conductivity sensor.

The device may comprise means for detecting the reactance of the bodily fluid, preferably in the gastrointestinal tract. The device may comprise means for detecting physical properties of the bodily fluid, such as viscosity.

It will be appreciated therefore that the device of the first aspect comprises one or more detection means. The device preferably has a size and shape which approximately resembles a capsule or pill, and which is readily ingestible by the subject without causing them pain or harm. The device may comprise processing means, for processing output data from the detection means. The device may comprise memory, such as a memory chip, in which output data from the detection means may be stored. The data may be downloaded from the memory, when the device is passed out of the body.

The device may comprise a power source, for example a battery. The device may comprise a printed circuit board (PCB) via which the detection means communicate with the processing means. In addition, the detection means require integrated circuitry to drive them and to measure the signals from them.

The device may comprise a transmitter for transmitting the output data from the detection means, either continuously or intermittently. The transmitter may transmit the output data using radio transmission, for example Wi-fi, Zigbee, Bluetooth or directional radio. It will be appreciated that the UK sets a range of different frequencies that can be used for transmission (for example 2.4 GHz for Wi-fi) without a licence. Also, different countries have different frequencies. Thus, the transmitter may transmit the output data at a frequency of at least 300 MHz, 500 MHz, 900 MHz, 1 GHz, 2 GHz, 2.4 GHz, 5.2/5.3/5.8 GHz, 10 GHz, 20 GHz, 24 GHz, or at least 60 GHz and above.

In embodiments where the output data are transmitted via a transmitter, the output data are preferably received by a receiver. The receiver is preferably remote from the diagnostic device. For example, the receiver may be attached to or worn by the subject. Advantageously, transmitting the data to the receiver means that the clinician is able to obtain real-time data corresponding to at least the gases and VOCs emitted from the subject, and so he can make an immediate diagnosis of the disease without having to wait for the device to pass along the subject's entire gastrointestinal tract. The inventors believe that the embodiment including the diagnostic device and the receiver is an important feature of the invention.

Hence, according to a second aspect, there is provided an apparatus for diagnosing disease, the apparatus comprise the diagnostic device of the first aspect, and a receiver.

Preferably, the receiver is arranged, in use, to receive output data transmitted by the transmitter. The receiver is preferably remote from the diagnostic device.

In a third aspect, there is provided use of the diagnostic device of the first aspect or the apparatus of the second aspect, for diagnosing disease in a subject.

In a fourth aspect, there is provided a method of diagnosing disease in a subject, the method comprising: (i) administering, to a subject requiring diagnosis, an ingestible diagnostic device comprising detection means for detecting gases and/or volatile organic compounds (VOCs); (ii) detecting gases and/or VOCs emitted by the subject by the detection means; and (iii) providing a diagnosis based on the detected gases and/or VOCs.

The method may comprise use of the device of the first aspect, or the apparatus of the second aspect. In use, as the device proceeds along the gastrointestinal tract of the subject, it detects output data corresponding to variables measured by the one or more detecting means, the position sensing means or the camera. In one embodiment, the method may comprise allowing the device to pass along the subject's entire gastrointestinal tract, and as it does so, it continuously or intermittently records data until it passes out of the subject. The memory chip may be recovered and output that has been stored in the memory chip may be downloaded, and analysed with software. A clinician may then be able to diagnose the disease based on the values of VOCs and gases detected by the detection means, including their type and concentration.

In another embodiment, the method may comprise continuously or intermittently transmitting output data from the one or more detection means by the transmitter, as the device passes along the subject's gastrointestinal tract. In this embodiment, the method may comprise receiving the output data by a receiver. Hence, the clinician can obtain real-time data corresponding to the gases and VOCs emitted by the patient, in addition to real-time information concerning the position of the device in the subject, real-time images from the camera, as well as immediate information corresponding to pH, temperature, dissolved oxygen concentration and/or thermal conductivity. The device will eventually be passed out of the subject, at which point the method comprises recovering the device and downloading and analysing the data stored in the memory chip.

The use of the third aspect or the method of the fourth aspect may be used to detect a wide range of diseases including, but not limited to, gastrointestinal disease, chronic liver disease, and pulmonary, localised or systemic infections. In addition, various metabolic diseases may be diagnosed, such as diabetes, obesity or impaired glucose tolerance. These conditions may reflect systemic changes of VOC profile originating in the gut, but manifesting disease in other organs. The device and apparatus may also be used to monitor treatment and recovery of diseases, as well as for assessing disease flair-up.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

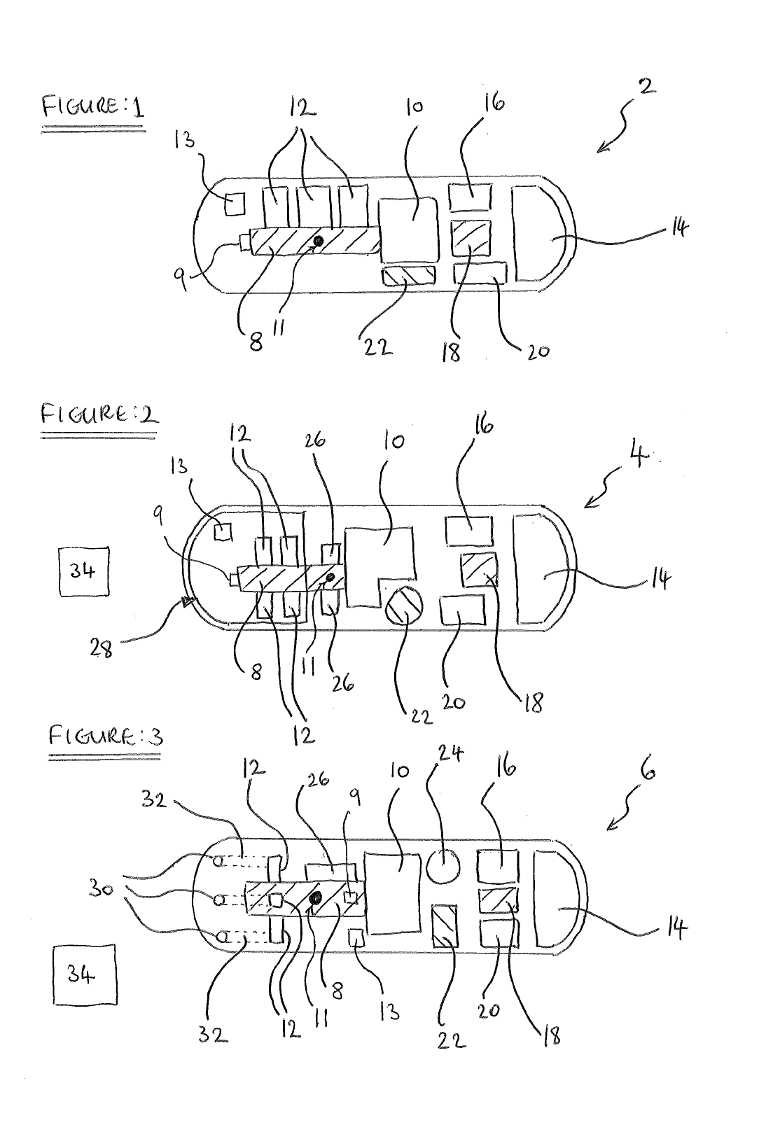

FIG. 1 is a schematic view of a first embodiment of an ingestible device according to the invention;

FIG. 2 is a schematic view of a second embodiment of the ingestible device; and

FIG. 3 is a schematic view of a third embodiment of the ingestible device.

### EXAMPLE 1

Referring to FIGS. 1-3, there are shown various embodiments of an ingestible device 2, 4, 6 according to the invention. The device 2, 4, 6 has the shape and dimensions of a standard pharmaceutical capsule or pill, and can be used to detect gases and volatile organic compounds (VOCs) emitted by a subject suffering from a disease, which is to be diagnosed. In use, the device 2, 4, 6 is first ingested by the subject, and then allowed to pass through the digestive tract during which time it detects the gases and VOCs being emitted by the subject. A clinician is able to diagnose the patient's disease by assessing the gases and VOCs that are being emitted, as will be discussed in detail below.

Referring to FIG. 1, the first embodiment of the device 2 is about 12 mm in length and about 5 mm in diameter. The device 2 has a central printed circuit board (PCB) 8 with integrated circuits that include a processor 9, which controls the device's 2 functions, and a memory chip 11. The device 2 is powered by a power source 10, for example a disc battery or miniature battery of 1.5V-24 V.

The device 2 includes one or more chemical sensors 12, which are capable of detecting gas and/or volatile organic chemicals (VOCs) emitted by the gastrointestinal tract of the patient. The sensors 12 need circuitry to drive them and to measure signals from them. In the embodiment shown in FIG. 1, the sensors 12 are disposed on or towards the surface of the device 2. The gaseous emissions are detected by sensors 12 using a variety of different technologies, for example:—
(i) resistive metal oxide (e.g. doped/undoped SnO₂), manufactured, for example, by Figaro, FIS or e2v based in Japan;
(ii) resistive mixed metal oxide e.g. combinations of SnO₂, WO₃, ZnO;
(iii) electrochemical gas sensors (available from Alphasense & City Technology);
(iv) resistive/capacitive/frequency measurement of conducting polymers (e.g. polypyrrole or polyaniline doped with a counter ion of DSA or BSA);
(v) resistive/capacitive/frequency measurement of composite polymers (e.g. carbon black nanoparticles dispersed in a polymer matrix of for example, polyethylene glycol), as previously produced by Smiths Detection;
(vi) optical measurement using infra-red (e.g. LED or some other IR-source, light filter with a photodetector (available from e2v or Infra-tec);
(vii) frequency measurement quartz crystal micro-balances/shear horizontal surface acoustic wave sensors (e.g. lithium niobate or lithium tantalite);
(viii) gas thermal measurement using pellisters/calorimetric (e.g. catalytic coating (e.g. palladium or platinum) of a bead formed from alumina oxide), manufactured by Figaro, FIS, e2v, or City Technology, amongst others; and
(ix) thermal techniques using a thermal conductivity sensor and/or a bio-sensor (e.g. an enzyme or protein attached to a secondary transducer).

In addition to detecting the emitted VOCs and gases by the sensors 12, the device 2 also includes a detecting unit 13, which detects the position of the device 2 when ingested by the subject. This is achieved through triangulation of the pill, by radio direction finding, employing, for example, the Doppler effect (or pseudo-Doppler), or an alternative magnetic tracking device.

The device 2 also includes a camera 14, which is connected to the PCB 8, processor 9 and memory chip 11. The camera 14 takes still images and/or video footage using either complementary metal oxide semi-conductor (CMOS) or charge-coupled device (CCD) camera technology illuminated by white or blue LEDs (not shown). The camera 15 can take pictures and video either simultaneously or serially with the gaseous/vapour measurements which are taken by the chemical sensors 12, and is powered by the power source 10 and/or energy scavenging technology, based on thermal gradients within the body or movement (e.g. spring technology commonly employed in wrist watches). Output from the camera 14 is processed by the processor 9, and stored on the memory chip 11.

In addition to the VOC/gas sensors 12 and the camera 14, the device 2 also includes a pH meter 16, a thermometer 18, a dissolved oxygen probe 20 and a thermal conductivity sensor 22. These sensors 16, 18, 20, 22 are arranged around the device 2, either internally or externally, and are provided to measure a range of different variables, as the device passes through the subject's gastrointestinal tract. The sensors 16, 18, 20, 22 are all connected to the printed circuit board 8 via integrated circuitry, and so the output data signals from each are stored in the memory chip 11 and/or processed by the processor 9. It will be appreciated that the device 2 can include any combination, or even all, of these additional sensors 16, 18, 20, 22 or the camera 14 or the detecting unit 13. However, in a basic embodiment, the device 2 only includes the VOC/gas sensors 12.

In use, as the device 2 proceeds along the gastrointestinal tract of the subject, it detects output data corresponding to the variables measured by the detecting unit 13, the sensors 12, 16, 18, 20, 22 and the camera 14, and stores these data in memory chip 11. In the embodiment shown in FIG. 1, the device 2 is allowed to pass along the subject's entire gastrointestinal tract, and as it does so, it continuously or intermittently records data until it passes out, at which point it is then located in the subject's waste. The memory chip 11 of the recovered device 2 is then connected to a PC (not shown), and the data that has been stored on the chip 11 is then downloaded, and analysed with software. Based on the values of VOCs and gases detected by the sensors 12, including their type and concentration, a clinician is then able diagnose the disease.

Referring to FIG. 2, there is a shown a second embodiment of the device 4. The device 4 has many of the features in the first embodiment of the device 2, including the PCB 8, processor 9, memory chip 11, battery 10, detecting unit 13, camera 14, and a range of peripheral sensors 16, 18, 20, 22. However, instead of being disposed on the surface of the device 2 (as shown in FIG. 1), in the embodiment shown in FIG. 2, the gas sensors 12 are disposed within a gas permeable membrane or packaging 28, which allows only gas and VOCs to pass therethrough, and prevents bodily fluids from passing therethrough. The gas permeable membrane 28, therefore, provides an effective barrier to the bodily fluids and, in certain sections of the gastrointestinal tract, body parts, which could otherwise interfere with the accurate detection of the gases and VOCs in the tract, as the device 4 passes therealong. The membrane 28 is porous/permeable, and may be one which is available under the trade name Gore-Tex or Vacol, from Dupont. It is possible to create the membrane 28 by controlling the hydrophilic/hydrophobic nature and micro-porosity of the material used. Gases are able to pass through the membrane 28 into a small area inside the device 4 where the sensors 12 would interact with gas.

As shown in FIG. 2, the device 4 also includes an aerial or transmitter 26 connected to the PCB 8. Hence, in this embodiment, the device 2 continuously or intermittently transmits the data stored in the memory chip 11 via the transmitter 26, as the device 2 passes along the subject's gastrointestinal tract. The transmitter 26 can transmit the signals corresponding to the variables detected by the various sensors 12, 16, 18, 20, 22, the detecting unit 13 and the camera 14, using radio transmission, including Wi-fi, Zigbee, Bluetooth or directional radio, each of which will be known to the skilled person. For example, the transmitter 26 shown in the Figures transmits data at a frequency of 2.4 GHz.

In the embodiment where the data are transmitted via the transmitter 26, the apparatus further includes a receiver 34, which is capable of receiving the data signals transmitted by the transmitter 26. Hence, the clinician can obtain real-time data corresponding to the gases and VOCs emitted by the patient, in addition to real-time information concerning the position of the device 4 in the subject via the detecting unit 13, real-time images from the camera 14, as well as immediate information corresponding to pH, temperature, dissolved oxygen concentration and thermal conductivity. The clinician, therefore, is able to make an immediate diagnosis of the disease without having to wait for the device 4 to pass along the subject's entire gastrointestinal tract. Of course, the device 4 will eventually be passed out, at which point it will still be recovered by the clinician, and the data stored on the memory chip 11 can be downloaded and analysed, if desired.

Referring to FIG. 3, there is shown a third embodiment of the device 6. As with the previous embodiments 2, 4, this embodiment includes many of the same features, including the PCB 8, processor 9, memory chip 11, battery 10, detecting unit 13, camera 14, a range of additional sensors 16, 18, 20, 22, a transmitter 26 and a detector 34. In addition, the device 6 includes an additional sensor 24, for measuring the reactance of the bodily fluid, similar to two probes of a multimeter. Alternatively, in another embodiment, sensor 24 can measure physical properties of the bodily fluid, such as viscosity, using a SAW device.

As shown in FIG. 3, the gas sensors 12 are disposed inside the device 6 instead of being on or towards the surface of the device 2 (as shown in FIG. 1), or contained within a gas permeable membrane 28 (as shown in FIG. 2). In order to feed or deliver gas and VOCs to the internal gas sensors 12, the device 6 includes a series of channels 32, one end of which are connected to an aperture 30 disposed on the outside of the device 6, and the other end of which is arranged such that it is at least adjacent the gas sensor 12. This arrangement forms a fluidic package. Hence, gases and VOCs emitted by the subject pass through aperture 30 and along channel 32, ultimately contacting gas sensors 12, for subsequent analysis. The sensors 12 are connected to the PCB 8, and the data can be processed by processor 9 and stored in the memory chip 11. The data are also transmitted via transmitter 26, and detected by detector 34.

### EXAMPLE 2

The various embodiments of the device 2, 4, 6 can be used to detect a range of diseases including, but not limited to, gastrointestinal disease, chronic liver diseases, pulmonary, localised and systemic infections. In addition, the device 2, 4, 6 can be also used to diagnose various metabolic diseases, such as diabetes, obesity or impaired glucose tolerance. These conditions may reflect systemic changes of dVOC profile originating in the gut but manifesting disease in other organs. The device 2, 4, 6 can also be used to monitor treatment and recovery of diseases, as well as for assessing disease flair-up.

For example, a subject may suffer from gastroenteritis, which is inflammation of the gastrointestinal tract, resulting in diarrhoea. The inflammation is frequently caused by an infection from certain viruses or bacteria, their toxins, parasites, or an adverse reaction to something in the diet or medication. Each of these micro-organisms emits a signature of various gases and VOCs, and so the detection of certain gases and VOCs by the device 2, 4, 6 is indicative of an infection with one or more of these micro-organisms. For example in Inflammatory Bowel Disease, ethanoic, butanoic, pentanoic acids, benzaldehyde, ethanal, carbon disulfide, dimethyldisulfide, acetone, 2-butanone, 2,3-butanedione, 6-methyl-5-hepten-2-one, indole, and 4-methylphenol have been identified as being significantly different compared with the corresponding levels in a healthy individual.

When a patient presents a large number of symptoms (e.g. altered bowel habit and systemic symptoms) to a clinician, one of the conditions that would need to be excluded is inflammatory bowel disease. As part of the rapid non-invasive diagnostic work-up, the patient is given one embodiment of the device 2, 4, 6 to ingest and an attached receiver 34, which is strapped to the body. The device 2, 4, 6 continuously transmits data corresponding to its location in the patient's alimentary canal via camera 14, as well as the various outputs from the sensors 16, 18, 20, 22, but also internally record the data for later download once the device 2, 4, 6 has been recovered. After 24-36 hours (depending on the gut transit time of the patient), the device 2, 4, 6 will be expelled naturally, and the data transmitted from the device 2, 4, 6, which was stored in the memory chip 11, will be analysed electronically using a PC, which runs statistical analysis and identification software, similar to that used in Pirouette & Multisens (statistical analysis packages used to process the data from analytical instruments).

Carrying out this process multiple times enables the construction of a model (such a Multi-layer perceptron and/or KNN, models that replicate some functions of the human brain, similar to neural networks), which will be tested against the existing data ‘chemical signature’ profile to identify the disease group(s), or to determine if the patient is in remission. Once confirmed, a rapid diagnosis is formulated which can then be utilised by the clinician who is then well-placed to initiate an appropriate treatment regime.

In certain instances, for example where toxic drugs are administered to the patient e.g. immuno-suppresives (Azathioprine, methotrexate, cyclosporin) and anti-cytokines (Infliximab, Adalumimab etc.), the device 2, 4, 6 can be used to rapidly determine if the VOC signatures profile has changed, either favourably or unfavourably. This information can then be used by the clinician to determine if they should continue or stop administering the potent drug to the patient.

In yet another embodiment, the device 2, 4, 6 includes a level of ‘intelligence’ with embedded analysis software, which is capable of suggesting and diagnosing the disease type. This has considerable benefits for the patient through avoiding several non-invasive tests, waiting times and rapid initiation of treatment (or withdrawal) with minimal disruption to the quality of life and time off work.

1. An ingestible diagnostic device comprising detection means for detecting gases and/or volatile organic compounds (VOCs).

2. A device according to claim 1, wherein the device is capable of detecting and characterising gases and other VOCs present in the gastrointestinal tract in vivo, for diagnosing disease.

3. A device according to claim 1, wherein the detection means is capable of detecting nitrous oxide, hydrogen sulphide, carbon dioxide, hydrogen ethanoic, butanoic and pentanoic acids, benzaldehyde, ethanal, carbon disulfide, dimethyldisulfide, acetone, 2-butanone, 2,3-butanedione, 6-methyl-5-hepten-2-one, indole and/or 4-methylphenol.

4. A device according to claim 1, wherein the detection means comprises one or more chemical sensors, which are capable of detecting gas and/or VOCs emitted from the gastrointestinal tract of the subject.

5. A device according to claim 1, wherein the detection means detects gas and/or VOC using technology selected from the group consisting of:
resistive metal oxide; resistive mixed metal oxide; electrochemical sensors;
resistive/capacitive/frequency measurement of conducting polymers;
resistive/capacitive/frequency measurement of composite polymers; optical measurement using infra-red; frequency measurement quartz crystal micro-balances/shear horizontal surface acoustic wave sensors; gas thermal measurement using pellisters/calorimetric technology; and thermal measurement techniques using a thermal conductivity sensor and bio-sensor.

6-7. (canceled)

8. A device according to claim 1, wherein the device comprises a gas permeable membrane or layer, which substantially surrounds the detection means.

9. A device according to claim 8, wherein the membrane is adapted to allow gas and VOCs to pass therethrough and reach the detection means, but prevents bodily fluids or solids from reaching the detection means.

10. (canceled)

11. A device according to claim 1, wherein the device comprises at least one channel, a first end of which is connected to an aperture disposed on the outer surface of the device, and a second end of which is arranged such that it is at least adjacent the detection means.

12. A device according to claim 11, wherein gas and VOCs emitted by the subject pass through the aperture and along the channel, such that it is fed to the detection means.

13. A device according to claim 1, wherein the device comprises position sensing means, which is capable of determining the location of the device when ingested by the subject.

14. A device according to claim 1, wherein the device comprises a camera, which is capable of taking still images and/or video footage.

15. A device according to claim 1, wherein the device comprises means for detecting pH and/or means for detecting temperature and/or means for detecting dissolved oxygen concentration.

16-17. (canceled)

18. A device according to claim 1, wherein the device comprises means for detecting thermal conductivity and/or means for detecting the reactance of the bodily fluid and/or means for detecting physical properties of the bodily fluid, such as viscosity.

19-20. (canceled)

21. A device according to claim 1, wherein the device comprises processing means for processing output data from the detection means, and wherein the device comprises memory, such as a memory chip, in which the output data from the detection means is stored.

22. (canceled)

23. A device according to claim 1, wherein the device comprises a power source, for example a battery.

24. A device according to claim 1, wherein the device comprises a transmitter for transmitting the output data from the detection means, either continuously or intermittently, and wherein the output data are received by a receiver.

25. (canceled)

26. An apparatus for diagnosing disease, the apparatus comprising the diagnostic device according to claim 1, and a receiver.

27. An apparatus according to claim 26, wherein the receiver is arranged, in use, to receive output data transmitted by the transmitter.

28. (canceled)

29. A method of diagnosing disease in a subject, the method comprising: (i) administering, to a subject requiring diagnosis, an ingestible diagnostic device comprising detection means for detecting gases and/or volatile organic compounds (VOCs); (ii) detecting gases and/or VOCs emitted by the subject by the detection means; and (iii) providing a diagnosis based on the detected gases and/or VOCs.

30. The method of claim 29, wherein the method comprises detecting gastrointestinal disease, chronic liver disease, or pulmonary, localised or systemic infections, diabetes, obesity or impaired glucose tolerance.
